(19) United States
(12) Patent Application Publication
(10) Pub. No.: US 2007/0264209 A1
(43) Pub. Date: Nov. 15, 2007

(54) Soluble containers for self-tanning formulations

(76) Inventors: Ratan K. Chaudhuri, Lincoln Park, NJ (US); Francois Marchio, Scarsdale, NY (US); Zoia Lascu, Valhalla, NY (US); Nadege Dumas Canedi, Highland Beach, FL (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/714,275

(22) Filed: Mar. 6, 2007

Related U.S. Application Data
(60) Provisional application No. 60/778,973, filed on Mar. 6, 2006.

(51) Int. Cl. A61K 8/18; A61K 31/19; A61K 31/21; A61K 31/715; A61K 36/04; A61K 36/752

(52) U.S. Cl. 424/59; 424/195.17; 424/736; 514/506; 514/557; 514/57

(57) ABSTRACT

There is provided a water-soluble container having a self-tanning agent sealed therein, a method for using said water-soluble container to form a self-tanning formulation and self-tanning formulations obtained therefrom. Methods for using the self-tanning formulations are provided as well as kits which include more than one water-soluble container.

### FIELD OF THE INVENTION

The present invention relates to soluble storage containers for self-tanning formulations and methods for their use.

### BACKGROUND OF THE INVENTION

Despite increasing public awareness of the risks of skin cancer and premature aging associated with sun exposure and the importance of sun protection, the notion that a tan promotes an attractive and healthy appearance remains strong. A tanned appearance can be maintained by the use of self-tanning products without sun exposure. A widely used self-tanning agent is dihydroxyacetone (DHA). In addition to the cosmetic browning effect provided by DHA application to the skin, the reaction product of DHA and the skin protein that produces the “tan” color is known to absorb UV light in 300-600 nm range, with a peak of about 350 nm. This protective effect against UV induced skin damage provides additional utilities for DHA as reported in the publications which follow:

1) Sunscreen agent (Johnson J A, Dihydroxyacetone for protection against long wavelength UVA radiation and blue light Brit J Dermatol, 1992 126:94);

2) Photoprotectant against UV-A, UV-B and visible light (Fusaro R M, and Johnson J A, Protection against long ultraviolet and/or visible light with topical dihydroxyacetone: Implications for the mechanism of action of the sunscreen combination, dihydroxyacetone/napthoquinone Dermatologica, 1975 150: 346-351; Petersen A B, Na R, Wulf H C, Sunless skin tanning with dihydroxyacetone delays broad-spectrum ultraviolet photocarcinogenesis in hairless mice Mutation Research, 2003 542:129-138);

3) Photoprotectant against erythema in humans (Faurschau A, Janjua N R, Wulf H C, Sun protection effect of Dihydroxyacetone Arch Dermatol, 2004 140:886-887);

4) Photoprotectant for uninvolved skin during psoralene-UV-A (PUVA) treatment of psoriasis (Taylor C R, Kwangsukstith C, Wimberly J, Kollias N, Anderson R R, Turbo-PUVA: dihydroxyacetone-enhanced photochemotherapy for psoriasis: a pilot study Arch Dermatol 1999 135:540-544);

5) Photoprotectant for patients suffering from erythropoietic protoporphyria (EPP) (Fusaro R M and Johnson J A, Dihydroxyacetone napthoquinone sunscreen J Am Med Assoc, 1972 222:1651-1657; Fusaro R M and Runge W J, Erythropoietic protoporphyria IV: Protection from sunlight Brit Med J, 1970 1:730-731); and

6) Photoprotectant for patients suffering from polymorphous light eruption (PLE) (Riccio G, Masturzo E, Del Dorbo A, Riccardo A M, Piccirillo M, Monfrecola G, Dermatite polimorfa solare: prevenzione mediante filtri UV e diidossiacetone G Ital Dermatol Venereol, 1999 134:197-200).

Publications which describe self-tanning products include:
Kurz T, Formulating effective self-tanners with DHA, Cosmet Toil, 1994 109:55-61;
Chaudhuri R K and Hwang C, Self-Tanners: Formulating with Dihydroxyacetone, Cosmet Toil, 2001 116:87-96;
Chaudhuri R K, Dihydroxyacetone: Chemistry and applications in self-tanning products, In Schlossman M, editor The Chemistry and Manufacture of Cosmetics, Allured Publishing, Vol III, Book One, 2002: pp 383-402;
Chaudhuri R K, Formulating with Dihydroxyacetone (DHA), In Surfactants in Personal Care and Decorative Cosmetics, 2nd edition, L D Rhein, M. Schlossman and A. O'Lenick, Eds. New York, Marcel Dekker, page 325-34, chapter 16, 2006;
U.S. Pat. No. 5,620,681, Tanaka S and Kenzo 1, Self-tanning cosmetic composition;
U.S. Pat. No. 5,612,044, Suares A J and Doblowski B J, Self-tanner cosmetic composition;

U.S. Pat. No. 5,922,333, Laughlin T J, System for automatically coating the human body;
US Publication No. 20060002870, Giacomoni, Paolo Ulderico and Manirazman, Abul M, Cosmetic compositions and methods containing a tanning agent and liposome encapsulated ursolic acid;
US Publication No. 20050238595, Stella, Qing, Morgan, Jeffrey Michael, Page, Steven Hardy, Kacher, Mark Leslie, Wei, Karl Shiqing, El-Nokaly, Magda, Personal care compositions that deposit sunless tanning benefit agents; and
US Publication No. 20050186236, Dorschner, Albrecht, Knuppel, Anja, Cosmetic and dermatological light protection formulations in the form of O/W macroemulsions or O/W microemulsions, with a content of dihydroxyacetone.

U.S. Pat. No. 5,351,831 discloses packaging certain materials, including certain liquids, in water-soluble bags consisting of a layer of cold water soluble polyvinyl alcohol.

Tanning products fall loosely into two categories-wash-offs and wear-offs. Wash-offs (also known as bronzers) are cosmetic tanning products in cream or gel formulations, typically without DHA, that give the appearance of a tan, but are not substantive to skin and wash-off in the shower at the end of the day. Wear-offs are self-tanning products containing DHA which, when applied to skin, develop a tan over a matter of hours that wears off over a period of five to ten days. These products are available in cream, lotion, milk, gel, foam, spray or airbrush formulations.

A popular vehicle used for self-tanning products is the emulsion. From an aesthetic viewpoint, emulsions are an elegant medium that can give the skin a smooth silky feel without being greasy or tacky and they can accommodate a wide variety of raw materials. DHA is most often formulated in oil/water emulsions.

Lotions are more popular than creams owing to their ease of spreadability on the skin and dispensability from bottles. Creams can lead to more intense tans than lotions because the applied film is thicker. Due to the high water solubility of DHA, aqueous or aqueous-alcoholic lotions and gels can be prepared easily. By appropriate control of viscosity, spray lotions or gels can also be prepared.

Sunless tanning booths employ misters to apply an even coat of a self-tanning product (solution) to the consumer's bare skin. In this mode of application, DHA is used as a solution in water or an aqueous-alcohol solution containing 8 to 15% DHA with other ingredients. The tanning booths employ misters that are either mobile or stationary, which help apply a uniform amount of self-tanning product (solution) to the body. Tanning sessions range from 6 to 60 seconds. Any configuration of the spray booth is suitable and spray methods which do not employ a booth are also suitable.

The self-tanning products employed in sunless tanning booths and other commercial systems require storage, handling and inventory of large amounts of DHA in solution. This is not a user-friendly way of handling DHA. Moreover, long-term storage of DHA in solution can cause degradation of DHA and may also require preservatives to avoid microbial contamination. More efficacious self-tanning products in solution, preferably, preservative free, are desired.

### SUMMARY OF THE INVENTION

The present invention provides a sealed water-soluble container with at least one self-tanning agent sealed therein, wherein the sealed container is comprised of a water-soluble polymer and/or copolymer.

The present invention also provides embodiments wherein a sealed water-soluble container of this invention is additionally sealed within a water-proof over wrap.

The present invention further provides a method of preparing a self-tanning formulation using a sealed water-soluble container of this invention. This method comprises dissolving a sealed water-soluble container of this invention in water or an aqueous- alcohol solution to form a sprayable solution or sprayable microemulsion of a self-tanning agent and a solubilized polymer and/or copolymer derived from the sealed water-soluble container.

The present invention further provides a self-tanning formulation which comprises a sprayable solution or sprayable microemulsion of a self-tanning agent and a solubilized polymer and/or copolymer such as polyvinyl acetate polymers, polyvinyl alcohol polymers and copolymers thereof. This solubilized polymer and/or copolymer will typically be derived from a sealed water-soluble container and this self-tanning formulation can be prepared by the method of this invention described above.

The present invention further provides a self-tanning formulation which comprises a sprayable solution or sprayable microemulsion of a self-tanning agent and a solubilized polymer and/or copolymer derived from a sealed water-soluble container and selected to enhance the color of the treated skin the color uniformity of the treated skin, the texture of the treated skin, and/or the rate at which the treated skin is colored.

The present invention further provides a self-tanning formulation which is free of preservatives.

The present invention additionally provides a method of tanning comprising topically applying to the skin, a self-tanning formulation of this invention.

The present invention also provides kits which comprise a water-soluble container of this invention and an additional water-soluble container with distinct and/or similar ingredients.

The sealed water-soluble containers, self-tanning formulations and kits of the present invention can “comprise” or “consist essentially of” or “consist of” the essential component(s). As used herein, the term “comprises” includes combinations of the recited essential component(s) with other ingredients without limitations. As used herein, “consisting essentially of” is a subgenus of “comprising” and means that the essential component(s) may include other ingredients, but only if the other ingredients do not materially alter the basic and novel characteristics of the essential component(s). It would be apparent to one skilled in the art what other common skin care ingredients can be incorporated in the containers formulation and kits of this invention without altering the substance of the containers formulation or kits. As used herein, “consists of” is a subgenus of “consisting essentially of” and means only the named essential component(s) is included.

The term “dermatologically-acceptable”, as used herein, means that the compositions or components thereof so described are suitable for use in contact with human skin without undue toxicity, incompatibility, instability, allergic response, and the like.

The containers formulations and kits of present invention comprise a tanning agent. Although any tanning agent known to those skilled in the art is within the scope of this invention, preferably dihydroxyacetone (DHA) is used. DHA is a known skin tanning agent. The benefits of packaging self-tanning agents, such as DHA, erythrulose and others, in a water-soluble bag include:
1) Accurate Pre-measured Unit Doses-Easy and Convenient to Use, no guess work and no measuring;
2) Elimination of Product Loss from Spills;
3) Preparation of Fresh Solution On-As-Needed Basis;
4) Improvement in Shelf-Life;
5) Eliminate the need for preservatives;
6) Environmentally Friendlier Packaging—water-soluble and biodegradable, no contamination of the water proof packages;
7) Safe Handling—Avoid Contact with Dry Product/Avoid Dust Inhalation; and
8) Reduced Storage Requirements and Economical Benefits in the Value Chain.

Current self-tanning formulations should normally be stored in cool conditions (<25° C.), and do not offer extended chemical stability. Hydrolytic instability of DHA is well documented, and leads to the formation of numerous undesirable side products such as formic acid, pyruvaldehyde and formaldehyde when stored or transported under standard conditions. (room temp; temperatures ranging from 25 to 40° C.)

A dry master batch ready to be dissolved right before application to skin minimizes the generation of side products and enhances the effectiveness. The packaging system enables the storage of DHA free from air and oxygen and those other factors responsible for the instability of DHA. The packaging system also eliminates the need for preservatives.

Water represents 50 to 90% of the finished sprayable formulation. Therefore, the present invention minimizes substantial warehousing and transportation costs. The final user can utilize city water right before application and has the capability to store the bags in reduced storage space.

As compared to other forms of transporting and handling dry goods, solid DHA in water-soluble bags eliminates any dusting problems which may arise from handling and shipping.

DHA may take several hours to effect a color change in the skin. The self-tanning formulation of this invention comprises a sprayable solution or sprayable microemulsion of a self-tanning agent and a solubilized polymer and/or copolymer. This solubilized polymer and/or copolymer can come from a sealed water-soluble container.

DHA in a water-soluble container, such as a bag, made of a water soluble polymer and/or copolymer, such as polyvinyl alcohol (PVA or PVOH), surprisingly provides a more natural looking tan at a more rapid rate when dissolved in water or an aqueous-alcohol solution and applied to skin.

It should be noted that other tanning agents are also contemplated to be within the scope of this invention. For example, tanning agents including but not limited to isatin, alloxan, ninhydrin, glyceraldehyde, glyceraldehyde phosphate, mesotartaric aldehyde, glutaraldehyde, erythrulose, erythrose, 6-aldo-D-fructose, 5-hydroxy-1,4-naphthochinon, 2-hydroxy-1,4-naphthochinon, hydroxymethylglyoxal, pyrazoline-4,5-dione derivatives, and 4,4-dihydroxypyrazoline-5 and derivatives thereof may used as an alternative or in conjunction with DHA and/or erythrulose.

In the preferred embodiments of the formulations, the tanning agent such as DHA is used in an amount of from 0. 1% to 25%, preferably from 0.5% to 20% and most preferably from 2% to 15% in solution.

The water-soluble containers used in the present invention can be in any configuration, such as bags, provided that they are comprised of a polymer and/or copolymer soluble in water or an aqueous-alcohol solution. Many types of water soluble polymers are suitable for use in accordance with the present invention. For example, suitable water soluble polymers include polyvinyl alcohol, polyoxyethylene, cellulose derivatives such as methylcellulose (MC), methylhydroxyethyl cellulose (MHEC), methylhydroxypropyl cellulose (MHPC), hydroxypropyl cellulose (HIPC), cellulose monoacetate, sodium polyacrylate, polylactic acid, lactic acid ethers or esters of polyvinyl alcohol, lactic acid ethers or esters of cellulose derivatives, lactic acid grafts on polyvinyl alcohol, carrageenan, pectin, combinations of the foregoing and combinations of the foregoing and starch. Commercially available PVA bags are supplied by Extra Packaging Corp of Rochester; Greensol of Sens, France; Monosol of Merriville, Ind.; and Gowan Milling LLC, of Yuma Ariz. A supplier of cellulosic bags is PFFC of Chicago, Ill.

Polyvinyl alcohol is a water-soluble synthetic polymer with excellent film-forming, emulsifying, and adhesive properties which is particularly suitable for use in the water soluble containers of this invention. Polyvinyl alcohol is prepared by polymerization of vinyl acetate. Partial or complete hydrolysis of this polymer provides polyvinyl alcohol. This versatile polymer offers outstanding resistance to oil, grease, and solvents, plus high tensile strength, flexibility, and high oxygen barrier. The basic properties of polyvinyl alcohol depend on its degree of polymerization, degree of hydrolysis, and distribution of hydroxyl groups.

In terms of the degree of polymerization, d.p. (based on the viscosity in centipoise (cP) of a 4 percent aqueous solution of polyvinyl alcohol at 20° C.), the major polyvinyl alcohol product groups are a low-viscosity group of approximately 5 cP, a medium viscosity group of 20-30 cP, and a high viscosity group of 40-50 cP, which correspond to d.p. of about 500, 1,700, and 2,000, respectively. In terms of degree of hydrolysis, the principal grades of polyvinyl alcohol produced can be classified as fully hydrolyzed (97.5-99.5 percent degree of hydrolysis) and partially hydrolyzed (87-89 percent degree of hydrolysis).

In general, fully hydrolyzed grades of polyvinyl alcohol are used mainly in paper coating, in textile warp sizing of hydrophilic fibers, such as cotton and rayon staple yarns, and in laminating film in safety glass (after conversion to polyvinyl butyral). On the other hand, partially hydrolyzed grades are used mainly in protective colloids within emulsions, in remoistenable adhesives, in textile warp sizing for rayon filaments and polyester fibers, and in printing plates. The most common grades in each area of application are fully and partially hydrolyzed grades with a degree of polymerization of about 1,700.

Polyvinyl alcohol is used mainly in aqueous solution. Its solubility in water depends on its degree of polymerization and degree of hydrolysis, with the latter factor being especially significant. The presence of as little as 2-3 mol percent of residual acetate groups causes a significant change in the solubility at 40-60° C.—fully hydrolyzed polyvinyl alcohol is only slightly soluble, but approximately 97 percent hydrolyzed polyvinyl alcohol is almost completely soluble.

Polyvinyl alcohol is one of the few truly biodegradable synthetic polymers; the degradation products are water and carbon dioxide.

A preferred water-soluble container comprises a polyvinyl alcohol bag. Since water-soluble bags are very hygroscopic, a water-proof outer wrap, preferably, high-density polyethylene, is desired for some embodiments.

Embodiments of this invention include a sealed water-soluble container of this invention additionally sealed within a water proof (insoluble) outer wrap. This outer wrap can be of any material that is not soluble in water or an aqueous-alcohol solution at 50° C. For example, suitable films are high density polyethylene, cellulose acetate lactate, cellulose lactate, methylhydroxybutyl cellulose, hydrophobically modified cellulose derivatives and combinations thereof. Other suitable materials for the outer wrap for this embodiment include acrylic acid polymers, acrylic ester polymers, acrylic acid copolymers, acrylic ester copolymers, copolymers of acrylic acids and esters, crosslinked polyvinyl alcohols, crosslinked cellulose derivatives, ethyl cellulose, nitrocellulose, cellulose butyrate, cellulose acetate butyrate, ethylene vinyl alcohol, ethylene vinyl acetate, polyvinylidene chloride and alkali soluble waxes and polyurethanes.

Preferred embodiments of the containers, formulations and kits of the present invention may contain one or more penetration enhancers to facilitate penetration of the composition through a water-soluble container, if needed. Suitable penetration enhancers include but are not limited to pentylene glycol, polysorbate-polyethylenesorbitan-monolaurate (Tween-20.RTM.), polyethylene glycol, ethoxydiglycol, dimethylsulfoxide, lecithin and mixtures thereof. The penetration enhancer is preferably used in an amount of from 0.001% to 10%, preferably from 0.01% to 8%, and most preferably from 0.1% to 5% by weight of the composition within the container.

The self-tanning formulation of this invention comprises a self-tanning agent, a solubilized polymer and/or copolymer and a dermatologically-acceptable vehicle that is suitable for topical application to skin, hair and/or nails. Dermatologically-acceptable vehicles are well known in the art and are selected based on the end use of the application. For example, vehicles of the present invention include, but are not limited to water, water-ethanol, water-ethylene glycol, water-glycerol etc. Such vehicles are well known to those of ordinary skill in the art, and can include one or more compatible liquid or solid filler diluents or vehicles which are suitable for application to the skin. The exact amount of vehicle will depend upon the level of any other optional ingredients that one of ordinary skill in the art would classify as distinct from the vehicle (e.g., other active components). The formulations, of the present invention preferably comprise from about 75% to about 99.99%, more preferably from about 85% to about 99.99%, and most preferably from about 93% to about 98%, by weight of the composition, of a vehicle.

The vehicle within the self-tanning formulations herein can be formulated in a number of ways to provide sprayable solutions or emulsions. For example, suitable emulsions include oil-in-water and water-in-oil emulsions. Preferred formulations comprise an oil-in-water emulsion. The solubilized polymer and/or copolymer is derived from a sealed water-soluble container and in certain embodiments, the polymer or copolymer is selected to enhance the color of the treated skin, the color uniformity of the treated skin, the texture of the treated skin and/or the rate at which the treated skin is colored.

The self-tanning formulations of this invention include embodiments which are free of preservatives which can help minimize or eliminate allergic reactions by those sensitive to conventional preservatives used in the cosmetic industry.

Preferred embodiments of the self-tanning formulations, the water-soluble containers and kits of the present invention may also contain humectants, which are conventionally used in this type of composition mainly to provide skin moisturization. Suitable moisturizers include but are not limited to polyols (namely, glycerine, sorbitol, propylene glycol, butylenes glycol), polyethylene glycols (PEG) and polysaccharides.

Further, the self-tanning formulations and kits of the present invention may include amino acids and/or amine potentiator, which comprise an amino-containing polymer. Suitable amino acids are glycine, alanin, histidine, or mixtures thereof. Suitable amino-containing polymers are amodimethicone, methoxy amodimethicone/silesquioxane copolymer, a linear polyethylenediamine, a branched polyethylenediamine, a polyethylenimine, a dendritic amino polymer, poly(lysine), poly(argine), or mixtures thereof. In order to prevent the reaction from occurring before application, the amino acids and/or amino-containing polymer must be included in a container, such as a water-soluble bag, which is different from the one containing the self-tanning agent.

The self-tanning formulation also can comprise other components/ingredients that may be chosen depending on the carrier and/or the intended use of the formulation. It should be noted that a person of ordinary skill in the art is capable of determining the amounts of additional components/ingredients that would be suitable for each formulation through routine experimentation. Additional components/ingredients, combined with DHA in the water-soluble bag or in a separate water-soluble bag to be mixed in a suitable vehicle prior to application, include, but are not limited to, antioxidants (such as sodium metabisulfite); chelating agents (such as disodium EDTA); emulsion stabilizers (such as carbomer); preservatives (such as methyl paraben); fragrances (such as pinene); flavoring agents (such as sorbitol); humectants (such as glycerol); waterproofing agents (such as PVP/Eicosene copolymer); water soluble film-formers (such as hydroxypropyl methylcellulose); oil-soluble film formers (such as hydrogenated C-9 Resin); moisturizing agents (such as glycerol, polyethylene glycols); cationic polymers (such as Polyquatenium 10); anionic polymers (such as xanthan gum); vitamins (such as tocopherol). Preferred additional components in the present invention include bronzers or other colorants that provide either an immediate tanned look, and/or provide the user with an indication of where the product has been applied so as to avoid streaking. Additionally preferred components include DHA-enhancing ingredients such as lactic acid, sugars, and film-forming agents.

Particularly preferred embodiments of the self-tanning formulations of present invention are sprayable solutions or lotions used as a self-tanning product. The present self-tanning formulations may therefore be combined with additional aesthetic agents such as moisturizers, emollients or humectants. Examples of useful combinations are oils, fats, waxes, esters, fatty acid alcohols, fatty acid ethoxylates, glycols, sugars, hyaluronic acid and hyaluronates, dimethicone, cyclomethicone, and the like. Further examples can be found in the International Cosmetic Ingredient Dictionary, CTFA, Ninth Edition, 2003.

The entire disclosures of all applications, patents and publications, cited herein are incorporated by reference herein.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

### EXAMPLES

The following examples describe sealed water-soluble containers of this invention, methods for their use and self-tanning formulations obtained therefrom.

### Example 1

Is a dual phase system comprised of two individual PVOH bags (PVOH=PVA) containing: a) the active mixture containing the self-tanning ingredient DHA and b) the additives mixture.

| | Typical Wt (%) |
|---|---|
| 1. Ingredients | |
| Dihydroxyacetone | 99 |
| Polyvinyl Alcohol Bag | 1 |
| 2. Ingredients | |
| Polyvinyl Alcohol Bag | 1 |
| Aloe Vera Powder | 10 |
| Sorbitol | 20 |
| Ectoin | 5 |
| PEG-32 | 35 |
| Erythrulose | 28 |
| Citric Acid | 1 |

### Example 2

Is a one phase system which is a polyvinyl alcohol (PVOH) bag containing the key active ingredient DHA along with penetration enhancers, humectants, moisturizers, pH adjuster, etc all in powder form.

| Ingredients | Typical Wt (%) |
|---|---|
| Dihydroxyacetone | 50 |
| Polyvinyl Alcohol Bag | 1 |
| Sorbitol | 14 |
| Aloe Vera Powder | 10 |
| PEG-32 | 20 |
| Ectoin | 5 |

### Example 3

Is a one-phase system incorporated in a polyvinyl alcohol (PVOH) bag containing the key active ingredient DHA along with penetration enhancers, humectants, moisturizers, non-ionic surfactants.

| Ingredients: | Typical Wt (%) |
|---|---|
| Dihydroxyacetone | 40 |
| Polyvinyl Alcohol Bag | 1 |
| Sorbitol | 10 |
| Polysorbate 60 | 5 |
| Oleth-5 | 1.5 |
| Ethanol | 20 |
| Aloe Vera Powder | 4 |
| PEG-32 | 18.50 |

Sprayable Finished Formulations:

### Example 4

Is a sprayable self-tanning solution (10% DHA) of this invention that may be obtained by dissolving 100 grams of each of the phases (a & b) which are described in Example 1 in 800 g liter of water (room temperatures) yielding 1 kilograms of finished solution.

| Ingredients | Typical Wt (%) |
|---|---|
| Water | 80 |
| Dihydroxyacetone | 10 |
| Polyvinyl Alcohol | 0.2 |
| Aloe Vera Powder | 1 |
| Sorbitol | 2 |
| Ectoin | 0.5 |
| PEG-32 | 3.5 |
| Erythrulose | 2.8 |
| Citric Acid | 0.01 |

### Example 5

Is a sprayable self-tanning microemulsion (10% DHA) of this invention that may be obtained by dissolving 100 grams of the product described in Example 3 in 300 g liter of water (room temperatures) yielding 400 g of finished product.

| Ingredients: | Typical Wt (%) |
|---|---|
| Water | 75 |
| Dihydroxyacetone | 10 |
| Polyvinyl Alcohol Bag | 0.25 |
| Sorbitol | 2.25 |
| Polysorbate 60 | 1.5 |
| Oleth-5 | 0.5 |
| Ethanol | 5 |
| Aloe Vera Powder | 1 |
| PEG-32 | 4.50 |

### Example 6

The composition prepared as in Example 4 was applied on human skin (lower arm, 6×6 cm² surface area) and compared with a similar composition prepared without polyvinyl alcohol. Visual observations were made at intervals of 1 hour for a period of 3 hours. Under these conditions, significant differences in color intensity and uniformity were noticed in the area treated with the 10% DHA bag solution, in contrast with a weaker, non-uniform color which resulted from the 10% DHA solution only.

The preceding examples can be repeated with similar success by substituting the generically or specifically described reactants and/or operating conditions of this invention for those used in the preceding examples.

1. A sealed water-soluble container with at least one self-tanning agent sealed therein, wherein the sealed water-soluble container is comprised of a water-soluble polymer and/or copolymer.

2. A sealed water-soluble container as in claim 1, which is a sealed bag comprised of a water-soluble polymer and/or copolymer.

3. A sealed water-soluble container as in claim 1, wherein the water-soluble polymer is polyvinyl alcohol (PVA), polyvinyl acetate (PVAc) or a combination thereof and the water-soluble copolymer is comprised of PVA and PVAc segments.

4. A sealed water-soluble container as in claim 3, which will dissolve completely in water or an aqueous-alcohol solution at temperatures of 10° C. and above.

5. A sealed water-soluble container as in claim 4, wherein the water-soluble polymer is partially hydrolyzed polyvinyl alcohol.

6. A sealed water-soluble container as in claim 3, which will dissolve completely in water or an aqueous-alcohol solution at temperatures of 60° C. and above and is stable in water at temperatures below of 40° C.

7. A sealed water-soluble container as in claim 3, which will dissolve completely in water or an aqueous-alcohol solution at temperatures of 90° C. and above and is stable in water at temperatures below 60° C.

8. A sealed water-soluble container as in claim 7, wherein the water soluble polymer is fully hydrolyzed polyvinyl alcohol.

9. A sealed water-soluble container as in claim 1, wherein
the water soluble polymer is polyvinyl alcohol, polyoxyethylene, methylcellulose (MC), methylhydroxyethyl cellulose (MHEC), methylhydroxypropyl cellulose (MHPC), hydroxypropyl cellulose (HIPC), cellulose monoacetate, sodium polyacrylate, polylactic acid, lactic acid ethers, an ester of polyvinyl alcohol (e.g. PVAce), a cellulose derivative, carrageenan, pectin or a combination thereof; and
the water soluble copolymer is a combination of segments of polyvinyl alcohol, polyoxyethylene, methylcellulose (MC), methylhydroxyethyl cellulose (MHEC), methylhydroxypropyl cellulose (MHPC), hydroxypropyl cellulose (HIPC), cellulose monoacetate, sodium polyacrylate, polylactic acid, lactic acid ethers, an ester of polyvinyl alcohol (e.g. PVAce), a cellulosic derivative, carrageenan or pectin.

10. A sealed water-soluble container as in claim 1, wherein the self-tanning agent is dihydroxyacetone.

11. A sealed water-soluble container as in claim 1, wherein the self-tanning agent is dihydroxyacetone and erythrulose.

12. A sealed water-soluble container as in claim 1, wherein the self-tanning agent is isatin, alloxan, ninhydrin, glyceraldehyde, glyceraldehyde phosphate, mesotartaric aldehyde, glutaraldehyde, erythrulose, erythrose, 6-aldo-D-fructose, 5-hydroxy-1,4-naphthochinon, 2-hydroxy-1,4-naphthochinon, hydroxymethylglyoxal, pyrazoline-4,5-dione derivatives, and 4,4-dihydroxypyrazoline-5 alone or in conjunction with dihydroxyacetone and/or erythrulose.

13. A sealed water-soluble container as in claim 10, wherein the sealed water-soluble container is comprised of polyvinyl alcohol, polyoxyethylene or a combination thereof.

14. A sealed water-soluble container as in claim 1, wherein the contents of the sealed water-soluble container additionally comprise a moisturizing agent, a skin penetration enhancing agent, an alcohol, and/or a film-forming agent.

15. A sealed water-soluble container as in claim 1, wherein the contents of the sealed water-soluble container additionally comprise a penetration enhancing agent which is pentylene glycol, polysorbate-polyethylenesorbitan-monolaurate (Tween-20.RTM.), polyethylene glycol, ethoxydiglycol, dimethylsulfoxide, lecithin or mixtures thereof.

16. A sealed water-soluble container as in claim 1, wherein the contents of the sealed water-soluble container additionally comprise antioxidants; chelating agents; emulsion stabilizers; preservatives;); flavoring agents; humectants; waterproofing agents; water soluble film-formers; oil-soluble film formers; moisturizing agents; cationic polymers; anionic polymers or vitamins.

17. A sealed water-soluble container as in claim 1, wherein the contents of the sealed water-soluble container additionally comprise bronzers.

18. A sealed water-soluble container as in claim 1, wherein the contents of the sealed water-soluble container are free of preservatives.

19. A sealed water-soluble container as in claim 1, which is additionally sealed within a water-proof outer wrap.

20. A sealed water-soluble container as in claim 19, wherein the water-proof outer wrap is high density polyethylene, cellulose acetate lactate, cellulose lactate, methylhydroxybutyl cellulose, hydrophobically modified cellulose derivatives or a combination thereof.

21. A sealed water-soluble container as in claim 19, wherein the water-proof outer wrap seals at least one additional water-soluble container.

22. A sealed water-soluble container as in claim 21, wherein the contents of the additional sealed water-soluble container comprise one or more of a moisturizing agent, a skin penetration enhancing agent, an alcohol or a film-forming agent.

23. A self-tanning formulation in the form of a cream, lotion, milk, gel, foam, sprayable solution or sprayable microemulsion which comprises a dermatologically-acceptable aqueous carrier, a self-tanning agent and either:
A) a water soluble polymer which is polyvinyl alcohol, polyoxyethylene, methylcellulose (MC), methylhydroxyethyl cellulose (MHEC), methylhydroxypropyl cellulose (MHPC), hydroxypropyl cellulose (HIPC), cellulose monoacetate, sodium polyacrylate, polylactic acid, lactic acid ethers, an ester of polyvinyl alcohol (e.g. PVAc), an ester of a cellulose derivative, a lactic acid graft on polyvinyl alcohol, carrageenan or pectin; or
B) a water soluble copolymer which is a combination of segments of polyvinyl alcohol, polyoxyethylene, methylcellulose (MC), methylhydroxyethyl cellulose (MHEC), methylhydroxypropyl cellulose (MHPC), hydroxypropyl cellulose (HIPC), cellulose monoacetate, sodium polyacrylate, polylactic acid, lactic acid ethers, an ester of polyvinyl alcohol(e.g. PVAc), an ester of a cellulose derivative, a lactic acid graft on polyvinyl alcohol, carrageenan or pectin.

24. A self-tanning formulation as in claim 23, prepared from a sealed water-soluble container with at least one self-tanning agent sealed therein, wherein the sealed water-soluble container is comprised of a water-soluble polymer and/or copolymer.

25. A self-tanning formulation as in claim 24, which is free of preservatives.

26. A self-tanning formulation as in claim 23, wherein the water-soluble polymer or copolymer is selected to enhance the color of the treated skin, enhance the color uniformity of the treated skin, enhance the texture of the treated skin, and/or enhance the rate at which the treated skin, is colored.

27. A kit comprising a sealed water-soluble container as in claim 1, and an additional water-soluble container with contents which comprise a moisturizing agent, a skin penetration enhancing agent, an alcohol, a film-forming agent, amino acids, an amino-containing polymer selected from amodimethicone, methoxy amodimethicone/silesquioxane copolymer, a linear polyethylenediamine, a branched polyethylenediamine, a polyethylenimine, a dendritic amino polymer, poly (lysine), poly(argine), or combination thereof.

28. A kit as in claim 27, wherein the sealed water-soluble containers are additionally sealed within a water-proof outer wrap comprised of high density polyethylene, cellulose acetate lactate, cellulose lactate, methylhydroxybutyl cellulose, hydrophobically modified cellulose derivatives or combinations thereof.

29. A method of effecting tanning on skin, said method comprising dissolving in water the sealed water-soluble container of claim 1 and applying the resultant aqueous solution to human skin.
